(19) United States
(12) Patent Application Publication
(10) Pub. No.: US 2008/0267888 A1
(43) Pub. Date: Oct. 30, 2008

(54) Heterocyclic Derivatives

(76) Inventors: Surendrakumar Satyanarayan Pandey, Chennai (IN); Gaddam Om Reddy, Chennai (IN); Gajendra Singh, Chennai (IN); Chitra Santhanagopalan, Chennai (IN); Sanjay Venkatachalapathi Kadnur, Chennai (IN); Debendranath Dey, Union City, CA (US); Abhijeet Nag, Union City, CA (US)

Correspondence Address: OLIFF & BERRIDGE, PLC, P.O. BOX 320850, ALEXANDRIA, VA, 22320-4850, US

(21) Appl. No.: 11/920,719

(22) PCT Filed: May 25, 2006
(86) PCT No.: PCT/IB2006/001364

§ 371 Date: May 23, 2008

(30) Foreign Application Priority Data
May 26, 2005 (IN) 635/CHE/2005

(51) Int. Cl. A61K 9/12; C07D 417/12; A61K 31/5377; A61P 29/00; C07D 413/12

(52) U.S. Cl. 424/45; 544/133; 514/230.8; 544/144

(57) ABSTRACT

The present invention relates to novel heterocyclic derivatives of the general formula (I), their pharmaceutically acceptable salts and compositions, their analogs, their tautomeric forms, and their stereoisomers. The present invention more particularly provides novel compounds of the general formula (I).

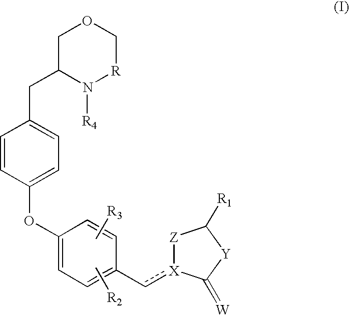

The following specification particularly describes the nature of the invention and the manner in which it has to be performed;

### FIELD OF THE INVENTION

The present invention relates to novel heterocyclic derivatives of the general formula (I), their pharmaceutically acceptable salts and compositions, their analogs, their tautomeric forms, and their stereoisomers. The present invention more particularly provides novel compounds of the general formula (I).

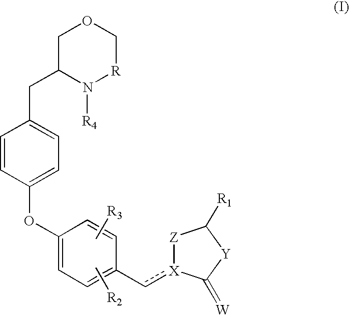

The present invention also relates to a process for the preparation of the above said novel compounds, their pharmaceutically acceptable salts and compositions, their analogs, their tautomeric forms, and their stereoisomers.

The compounds of the present invention are effective in lowering blood glucose, serum insulin, free fatty acids, cholesterol and triglyceride levels and are useful in the treatment and/or prophylaxis of type II diabetes. These compounds are effective in treatment of obesity, inflammation, autoimmune diseases such as multiple sclerosis and rheumatoid arthritis. Surprisingly, these compounds increase the leptin level and have no liver, toxicity.

Furthermore, the compounds of the present invention are useful for the treatment of disorders associated with insulin resistance, such as polycystic ovary syndrome, as well as hyperlipidemia, coronary artery disease, peripheral vascular disease, and for the treatment of inflammation and immunological diseases, particularly those mediated by cytokines such as TNF-α, IL-1, IL-6, IL-1β and cyclooxygenases such as COX-2.

### BACKGROUND OF THE INVENTION

The causes of type I and II diabetes are not yet clear, although both genetics and environment seem to be the factors. Type I diabetes is an autonomic immune disease and patient must take insulin to survive. Type II diabetes is the more common form, it is a metabolic disorder resulting from the body's inability to make a sufficient amount of insulin or to properly use the insulin that is produced. Insulin secretion and insulin resistance are considered to be the major defects; however, the precise genetic factors involved in the mechanism remain unknown.

Patients with diabetes usually have one or more of the following defects:

Less production of insulin by the pancreas;

Over secretion of glucose by the liver;

Independence of the glucose uptake by the skeletal muscles;

Defects in glucose transporters, desensitization of insulin receptors; and

Defects in the metabolic breakdown of polysaccharides.

Other than the parenteral or subcutaneous administration of insulin, there are about four classes of oral hypoglycemic agents used i.e. sulfonylureas, biguanides, alpha glucosidase inhibitors and thiazolidinediones. Each of the current agents available for use in treatment of diabetes has certain disadvantages. Accordingly, there is a continuing interest in the identification and development of new agents, which can be orally administered, for use in the treatment of diabetes.

The thiazolidinedione class listed above has gained more widespread use in the recent years for the treatment of type II diabetes, exhibiting particular usefulness as insulin sensitizers to combat “insulin resistance”, a condition in which the patient becomes less responsive to the effects of insulin. However there is a continuing need for nontoxic, more widely effective insulin sensitizers. In our continuous efforts to explore new compounds having antidiabetic activity, is our present invention where we propose to synthesize new compounds containing rhodanine, rhodanine-3-aceticacid, thiazolidinone, oxindole, benzathiazolone, morpholone, morpholine, and the oxazolidinone system and also study them for anti-diabetic activity by taking thiazolidinone as a comparator.

Recent advances in the scientific understanding of the mediators involved in acute and chronic inflammatory diseases and cancer have led to new strategies in the search for effective therapeutics. Traditional approaches include direct target intervention such as the use of specific antibodies, receptor antagonists, or enzyme inhibitors. Recent breakthroughs in the elucidation of regulatory mechanisms involved in the transcription and translation of a variety of mediators have led to an increased interest in the therapeutic approaches directed at the level of gene transcription.

As indicated above, the present invention is also concerned with the treatment of immunological diseases or inflammation, notably such diseases as are mediated by cytokines or cyclooxygenase. The principal elements of the immune system are macrophages or antigen-presenting cells, T cells and B cells. The role of other immune cells such as NK cells, basophils, mast cells and dendritic cells are known, but their role in primary immunologic disorders is uncertain. Macrophages are important mediators of both inflammation and provide the necessary “help” for T cell stimulation and proliferation. Most importantly macrophages make IL 1, IL 12 and TNF-α all of which are potent pro-inflammatory molecules and also provide help for T cells. In addition, activation of macrophages results in the induction of enzymes, such as cyclooxygenase II (COX-2), inducible nitric oxide synthase (iNOS) and production of free radicals capable of damaging normal cells. Many factors activate macrophages, including bacterial products, superantigens and interferon gamma (IFNγ). It is believed that phosphotyrosine kinases (PTKs) and other undefined cellular kinases are involved in the activation process.

Cytokines are molecules secreted by immune cells that are important in mediating immune responses. Cytokine production may lead to the secretion of other cytokines, altered cellular function, cell division or differentiation. Inflammation is the normal response of the body to injury or infection. However, in inflammatory diseases such as rheumatoid arthritis, pathologic inflammatory processes can lead to morbidity and mortality. The cytokine tumor necrosis factor-alpha (TNF-α) plays a central role in the inflammatory response and has been targeted as a point of intervention in inflammatory disease. TNF-α is a polypeptide hormone released by activated macrophages and other cells. At low concentrations, TNF-α participates in the protective inflammatory response by activating leukocytes and promoting their migration to extravascular sites of inflammation (Moser et al., J Clin Invest, 83, 444-55, 1989). At higher concentrations, TNF-α can act as a potent pyrogen and induce the production of other pro-inflammatory cytokines (Haworth et al., Eur J Immunol, 21, 2575-79, 1991; Brennan et al., Lancet, 2, 244-7, 1989). TNF-α also stimulates the synthesis of acute-phase proteins. In rheumatoid arthritis, a chronic and progressive inflammatory disease affecting about 1% of the adult U.S. population, TNF-α mediates the cytokine cascade that leads to joint damage and destruction (Arend et al., Arthritis Rheum, 38, 151-60, 1995). Inhibitors of TNF-α, including soluble TNF receptors (etanercept) (Goldenberg, Clin Ther, 21, 75-87, 1999) and anti-TNF-α antibody (infliximab) (Luong et al., Ann Pharmacother, 34, 743-60, 2000), have recently been approved by the U.S. FDA as agents for the treatment of rheumatoid arthritis. Elevated levels of TNF-α have also been implicated in many other disorders and disease conditions, including cachexia, septic shock syndrome, osteoarthritis, inflammatory bowel disease such as Crohn's disease and ulcerative colitis etc. It can be seen that inhibitors of TNF-α are potentially useful in the treatment of a wide variety of diseases. Compounds that inhibit TNF-α have been described in several patents.

Excessive production of IL-6 is implicated in several disease states; it is highly desirable to develop compounds that inhibit IL-6 secretion. Compounds that inhibit IL-6 have been described in the U.S. Pat. Nos. 6,004,813; 5,527,546 and 5,166,137.

The cytokine IL-1β also participates in the inflammatory response. It stimulates thymocyte proliferation, fibroblast growth factor activity, and the release of prostaglandin from synovial cells. Elevated or unregulated levels of the cytokine IL-1β have been associated with a number of inflammatory diseases and other disease states, including but not limited to adult respiratory distress syndrome, allergy, Alzheimer's disease etc. Since the overproduction of IL-1β is associated with numerous disease conditions, it is desirable to develop compounds that inhibit the production or activity of IL-1β.

It will be appreciated from the foregoing facts that, while there have been extensive prior efforts to provide compounds for inhibiting, for example, TNF-α, IL-1, IL-6, COX-2 or other agents considered responsible for immune response, inflammation or inflammatory diseases, e.g. arthritis, there still remains a need for new and improved compounds for effectively treating or inhibiting such diseases. With an objective of providing compounds, which are effective for such treatments as well as for the treatment of, for example, insulin resistance, hyperlipidemia, obesity, inflammation, multiple sclerosis and arthritis, we have continued our research to develop new thiazoldinediones along with other heterocyclic analogs.

Few Prior Art References, which Disclose the Closest Compounds, are Given Here:

i) WO 01/02377 discloses compounds of the formula (Ia) as telomerase inhibitors

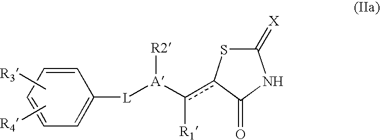

wherein R′₁ and R′₂ represents hydrogen, alkyl etc., X represents oxygen or sulfur; ---- is a single or double bond; L represents oxygen, nitrogen, sulfur; R′₃ represents hydrogen, alkyl, aryl etc., R′₄ represents hydrogen, alkyl, aryl etc., A′ represents aryl.

An example of these compounds is shown in formula (IIb)

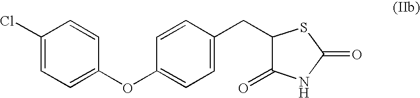

ii) EP 1148054 discloses compounds of formula (IIc)

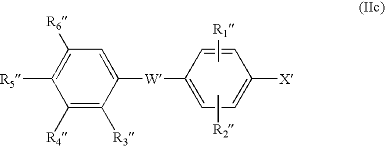

wherein R₁″, R₂″, R₃″, R₅ ″, R₆″, represent hydrogen, alkyl etc., X′ represents methylene thiazolidin-2,4-dione, methylene oxazolidin-2,4-dione etc., W′ represents oxygen, sulfur; R₄″ represents hydrogen, alkyl substituted with zero to three substituents etc.

An example of these compounds is shown in formula (IId)

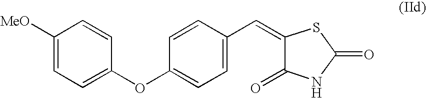

iii) U.S. Pat. No. 6,331,633 discloses compounds of formula (IIe)

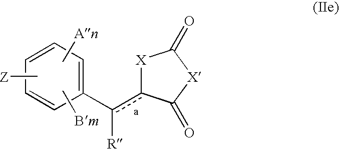

wherein Z is

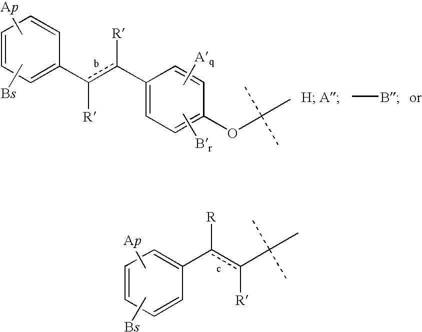

wherein n, m, q and r are independently integers from zero to 4; p and s are independently integers from zero to 5; a, b and c are double bonds which may be present or absent; R, R′ and R″ are independently H, C₁-C₂₀ linear or branched alkyl, C₂-C₂₀ linear or branched alkenyl, —CO₂H, —CO₂R′″, —NH₂, —NHR′″, —NR₂′″, —OH, —OR′″, halo, substituted C₁-C₂₀ linear or branched alkyl or substituted C₂-C₂₀ linear or branched alkenyl, wherein R′″ is C₁-C₂₀ linear or branched alkyl or linear or branched alkenyl; A, A′ and A″ are independently H, C₁-C₂₀acyl amino; C₁-C₂₀acyloxy; C₁-C₂₀alkanoyl; C₁-C₂₀alkoxycarbonyl; C₁-C₂₀alkoxy; C₁-C₂₀alkylamino; C₁-C₂₀alkylcarboxylamino; carboxyl; cyano; halo; hydroxy; B, B′ and B″ are independently H; C₁-C₂₀acylamino; C₁-C₂₀ acyloxy; C₁-C₂₀ alkanoyl; etc., X, X′ are independently —NH, —NR′″, O or S.

An example of these compounds is shown in formula (IIf)

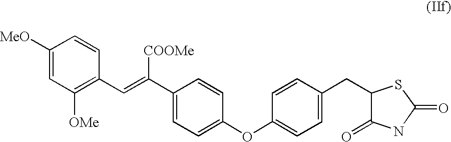

iv) Tetrahedron asymmetry 14, 2003, 2619-2623 discloses the two step synthesis of enantiopure tert-butyl (1S)-2-hydroxy-1-(4-benzyloxybenzyl)ethylcarbamate from N-Boc-L-tyrosine(1a).

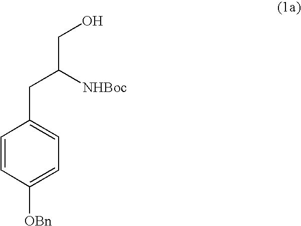

### OBJECTIVE OF THE INVENTION

With an objective of developing novel compounds for lowering the blood glucose, free fatty acids, cholesterol and triglyceride levels in type II diabetes and to treat autoimmune diseases such as multiple sclerosis and rheumatoid arthritis, we focused our research to develop new compounds effective in the treatment of the above mentioned diseases, and efforts in this direction have led to compounds having the general formula (I).

The main objective of the present invention is therefore, to provide heterocyclic derivatives of the general formula (I), their pharmaceutically acceptable salts and compositions, their analogs, their tautomeric forms, and their stereoisomers that are useful for the treatment of disorders associated with insulin resistance, such as polycystic ovary syndrome, as well as hyperlipidemia, coronary artery disease, peripheral vascular disease, and are also useful for the treatment of inflammation and immunological diseases, particularly those mediated by cytokines such as TNF-α, IL-1, IL-6, IL-1β and cyclooxygenases such as COX-2. Another objective of the present invention is to provide novel heterocyclic derivatives of the general formula (I), their pharmaceutically acceptable salts and compositions, their analogs, their tautomeric forms, and their stereoisomers having enhanced activities, without toxic effects or with reduced toxic effects. Yet another objective of the present invention is to provide a process for the preparation of the novel heterocyclic derivatives of the general formula (I), their pharmaceutically acceptable salts and compositions, their analogs, their tautomeric forms, and their stereoisomers.

### SUMMARY OF THE INVENTION

The present invention, relates to novel heterocyclic derivatives of the general formula (I)

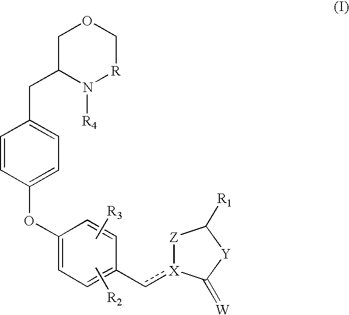

their pharmaceutically acceptable salts and compositions, their analogs, their tautomeric forms, and their stereoisomers; wherein ---- represents an optional bond; R represents CH₂, C═O; W represents O or S; X represents C, CH or N; Y represents NR₅, S or O, wherein R₅ represents hydrogen, substituted or unsubstituted alkyl, alkenyl, —CH₂COOR′, aryl, or a counter ion; wherein R′ represents H or an alkyl group; Z represents CR₆ or S; R₁ represents ═O, ═S or together with R₆ forms a fused 5 or 6 membered aromatic or heteroaromatic ring system containing carbon atoms or 1 or 2 heteroatoms selected from O, S or N; R₂ and R₃ may be same or different and independently represent hydrogen, halogen, hydroxy, nitro, cyano, formyl, amino, alkyl, haloalkyl, alkoxy group; R₄ represents H, COR₇, substituted or unsubstituted groups selected from alkyl, alkenyl, aryl, aryloxy, alkoxy, heteroaryl or heterocyclyl; wherein R₇ represents H, substituted or unsubstituted groups selected from alkyl, alkenyl, aryl, aryloxy, alkoxy or aralkoxy.

### DETAILED DESCRIPTION OF THE INVENTION

Suitable groups represented by R represent CH₂, C═O;

R₁ is selected from ═O, ═S; or together with R₆ forms a fused 5 or 6 membered aromatic or heteroaromatic ring system containing carbon atoms or 1 or 2 heteroatoms selected form O, S or N such as phenyl, naphthyl, furyl, pyrrolyl, pyridyl and the like.

Suitable groups represented by R₂ and R₃, are selected from hydrogen, halogens such as fluorine, chlorine, bromine or iodine; hydroxy, nitro, cyano, formyl, amino, substituted or unsubstituted linear or branched (C₁-C₄) allyl groups such as methyl, ethyl, propyl, isopropyl, n-butyl, isobutyl, t-butyl, and the like; haloalkyl groups selected from alkyl group substituted by one, two, three or four halogen atoms such as chloromethyl, chloroethyl, trifluoromethyl, trifluoroethyl, dichloromethyl, dichloroethyl and the like; substituted or unsubstituted (C₁-C₄) alkoxy group such as methoxy, ethoxy, propoxy, butoxy and the like.

Suitable groups represented by R₄ are selected from hydrogen, substituted or unsubstituted groups selected from (C₁-C₄) alkyl groups such as methyl, ethyl, propyl, isopropyl, n-butyl, isobutyl, t-butyl and the like; substituted or unsubstituted linear or branched (C₂-C₇) alkenyl groups such as ethenyl, propenyl, butenyl and the like; aryl groups such as phenyl, naphthyl and the like, the aryl group may be substituted; aryloxy groups such as phenoxy, napthoxy and the like, substituted or unsubstituted linear or branched (C₂-C₄) alkoxy groups such as methoxy, ethoxy, propoxy, n-butoxy, and the like; heteroaryl groups such as pyridyl, thienyl, furyl, pyrrolyl, oxazolyl, thiazolyl, imidazolyl, oxadiazolyl, triazolyl, tetrazolyl, pyrimidinyl, pyrazinyl, indolyl, indolinyl, benzothiazolyl, and the like, which may be substituted; heterocyclyl groups such as pyrrolidinyl, thiazolidinyl, oxazolidinyl, morpholinyl, thiomorpholinyl, piperidinyl, piperazinyl, and the like, which may be substituted, COR₇, wherein R₇ represents H; substituted or unsubstituted groups selected from (C₁-C₄) alkyl groups such as methyl, ethyl, propyl, isopropyl, n-butyl, isobutyl, t-butyl and the like; substituted or unsubstituted linear or branched (C₂-C₇) alkenyl groups such as ethenyl, propenyl, butenyl and the like; aryl groups such as phenyl, naphthyl and the like, the aryl group may be substituted; aryloxy groups such as phenoxy, napthoxy and the like; substituted or unsubstituted linear or branched (C₂-C₂₀) alkoxy groups such as methoxy, ethoxy, propoxy, n-butoxy, isobutoxy, t-butoxy and the like;

Pharmaceutically acceptable salts of the present invention include base addition salts such as alkali metal salts like Li, Na, and K salts, alkaline earth metal salts like Ca and Mg salts, salts of organic bases such as diethanolamine, α-phenylethylamine, benzylamine, piperidine, morpholine, pyridine, hydroxyethylpyrrolidine, hydroxyethylpiperidine, choline and the like, ammonium or substituted ammonium salts, aluminum salts. Salts also include amino acid salts such as glycine, alanine, cystine, cysteine, lysine, arginine, phenylalanine, guanidine etc. Salts may include acid addition salts where appropriate which are sulphates, nitrates, phosphates, perchlorates, borates, hydrohalides, acetates, tartrates, maleates, citrates, succinates, palmoates, methanesulphonates, tosylates, benzoates, salicylates, hydroxynaphthoates, benzenesulfonates, ascorbates, glycerophosphates, ketoglutarates and the like. Pharmaceutically acceptable solvates may be hydrates or comprising of other solvents of crystallization such as alcohols.

The protecting groups used in the invention are conventional protecting groups such as t-butoxycarbonyl(t-Boc), trityl, trifluoroacetyl, benzyloxy, benzyloxy carbonyl(Cbz) and the like and deprotection can be done by conventional methods.

### Particularly Useful Compounds According to the Invention Include:

5-(4-{4-[(5-oxomorpholin-3-yl)methyl]phenoxy}benzylidene)-1,3-thiazolidine-2,4-dione;
5-(4-{4-[(4-oxo-2-thioxo-1,3-thiazolidin-5-ylidene)methyl]phenoxy}benzyl)morpholin-3-one;
5-(-4-{4-[(5-oxomorpholin-3-yl)methyl]phenoxy}benzylidene)-4-oxo-2-thioxo-1,3-thiazolidin-3-yl]acetic acid;
5-(4-{4-[(5-oxomorpholin-3-yl)methyl]phenoxy}benzylidene)-1,3-dihydro-2H-indol-2-one;
5-(3-fluoro-4-{4-[(5-oxomorpholin-3-yl)methyl]phenoxy}benzylidene)-1,3-thiazolidine-2,4-dione;
5-(3-fluoro-4-{4-[(5-oxomorpholin-3-yl)methyl]phenoxy}benzylidene)-4-oxo-2-thioxo-1,3-thiazolidin-3-yl)acetic acid;
5-(3-chloro-4-{4-[(5-oxomorpholin-3-yl)methyl]phenoxy}benzylidene)-1,3-thiazolidine-2,4-dione;
5-(4-{2-chloro-4-[(4-oxo-2-thioxo-1,3-thiazolidin-5-ylidene)methyl]phenoxy}benzyl)morpholin-3-one;
5-(3-chloro-4-{4-[(5-oxomorpholin-3-yl)methyl]phenoxy}benzylidene)-1,3-dihydro-2H-indol-2-one;
5-(2-chloro-4-{4-[(5-oxomorpholin-3-yl)methyl]phenoxy}benzylidene)-4-oxo-2-thioxo-1,3-thiazolidin-3-yl]acetic acid;
5-(4-{3-chloro-4-[(4-oxo-2-thioxo-1,3-thiazolidin-5-ylidene)methyl]phenoxy}benzyl)morpholin-3-one;
5-(2-chloro-4-{4-[(5-oxomorpholin-3-yl)methyl]phenoxy}benzylidene)-1,3-thiazolidine-2,4-dione;
5-[4-{4-[(5-oxomorpholin-3-yl)methyl]phenoxy}-3-(trifluoromethyl) benzylidene]-1,3-thiazolidine-2,4-dione;
5-(2-chloro-4-{4-[(5-oxomorpholin-3-yl)methyl]phenoxy}benzylidene)-1,3-dihydro-2H-indol-2-one;
5-(3-fluoro-4-{4-[(5-oxomorpholin-3-yl)methyl]phenoxy}benzylidene)-1,3-dihydro-2H-indol-2-one;
5-(4-{2-fluoro-4-[(4-oxo-2-thioxo-1,3-thiazolidin-5-ylidene)methyl]phenoxy}benzyl)morpholin-3-one;
5-(3-(trifluoromethyl)-4-{4-[(5-oxomorpholin-3-yl)methyl]phenoxy}benzylidene)-1,3-dihydro-2H-indol-2-one;
5-(4-{4-[(4-oxo-2-thioxo-1,3-thiazolidin-5-ylidene)methyl]-2-(trifluoromethyl)phenoxy}benzyl)morpholin-3-one;
5-(4-{4-[(4-methyl-5-oxomorpholin-3-yl)methyl]phenoxy}benzylidene)-1,3-thiazolidine-2,4-dione;
4-methyl-5-(4-{4-[(4-oxo-2-thioxo-1,3-thiazolidin-5-ylidene) methyl]phenoxy}benzyl)morpholin-3-one;
5-(4-{2-methoxy-4-[(4-oxo-2-thioxo-1,3-thiazolidin-5-ylidene)methyl]phenoxy}benzyl)morpholin-3-one;
5-(3-trifluoromethyl-4-{4-[(5-oxomorpholin-3-yl)methyl]phenoxy}benzylidene)-4-oxo-2-thioxo-1,3-thiazolidin-3-yl]acetic acid;
5-(4-{3-fluoro-4-[(4-oxo-2-thioxo-1,3-thiazolidin-5-ylidene)methyl]phenoxy}benzyl)morpholin-3-one;
5-(3-fluoro-4-{4-[(4-methyl-5-oxomorpholin-3-yl)methyl]phenoxy}benzylidene)-1,3-thiazolidine-2,4-dione;
5-(2-fluoro-4-{4-[(5-oxomorpholin-3-yl)methyl]phenoxy}benzylidene)-1,3-dihydro-2H-indol-2-one;
5-(2-fluoro-4-{4-[(5-oxomorpholin-3-yl)methyl]phenoxy}benzylidene)-4-oxo-2-thioxo-1,3-thiazolidin-3-yl]acetic acid;
5-(3-fluoro-4-{4-[(4-methyl-5-oxomorpholin-3-yl)methyl]phenoxy}benzylidene)-4-oxo-2-thioxo-1,3-thiazolidin-3-yl]acetic acid;
5-(4-{2-chloro-4-[(4-oxo-2-thioxo-1,3-thiazolidin-5-ylidene)methyl]phenoxy}benzyl)-4-methylmorpholin-3-one;
5-(2-chloro-4-{4-[(4-methyl-5-oxomorpholin-3-yl)methyl]phenoxy}benzylidene)-1,3-thiazolidine-2,4-dione;
5-(4-{3-chloro-4-[(4-oxo-2-thioxo-1,3-thiazolidin-5-ylidene)methyl]phenoxy}benzyl)-4-methylmorpholin-3-one;
5-(3-chloro-4-{4-[(4-methyl-5-oxomorpholin-3-yl)methyl]phenoxy}benzylidene)-1,3-thiazolidine-2,4-dione;
5-(4-{4-[(5-oxomorpholin-3-yl)methyl]phenoxy}benzyl)-1,3-dihydro-2H-indol-2-one;
5-(3-fluoro-4-{4-[(5-oxomorpholin-3-yl)methyl]phenoxy}benzyl)-1,3-thiazolidine-2,4-dione;
5-(4-{2-chloro-4-[(4-oxo-2-thioxo-1,3-thiazolidin-5-yl)methyl]phenoxy}benzyl)morpholin-3-one.

Preferred salts for the list of compounds given above are hydrochloride, hydrobromide, sodium, potassium or magnesium.

In another aspect the invention provides novel pharmaceutical compositions comprising the heterocyclic derivatives of the formula (I) as set out above. The said compositions may comprise the heterocyclic derivatives as the active ingredient together with the pharmaceutically acceptable carrier, diluent or excipient. The composition may be prepared by processes known in the art and may be in the form of a tablet, capsule, powder, syrup, solution or suspension. The amount of the active ingredient in the composition may be less than 60% by weight.

According to another feature of the present invention, there is provided a process for the preparation of the compounds of formula (I) wherein ---- represents a bond, and all the other symbols are as defined earlier, as shown in the scheme:

### Scheme:

a) Deprotection of the compound of formula (1a) to (2a) and further alkylation of the compound of formula (2a) gave the compound of formula (3a). The compound of formula (1 a) is prepared according to the procedure described in Tetrahedron asymmetry 14, 2619-2623 (2003).

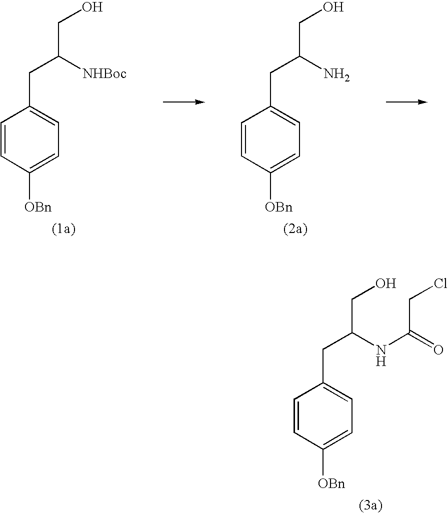

b) Cyclization of the compound of formula (3a) gave (4a); which is further debenzylated to give the compound of formula (5a) wherein all other symbols are as defined earlier. Alternatively the compound of formula (4a) wherein R₄=alkyl can be prepared by alkylation of compound of formula (4a) wherein R₄=hydrogen by conventional methods.

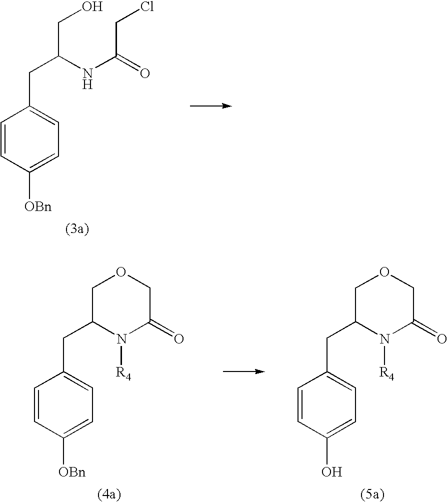

b) Optional reduction of the compound of formula (5a) gave the compound of formula (6a) wherein all the symbols are as defined earlier.

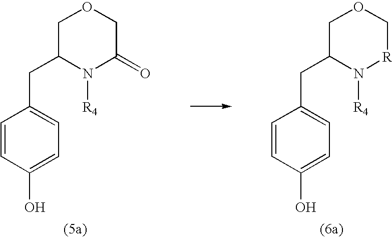

c) Condensation of the compound of formula (6a) with a compound of formula (7a) gave the compound of formula (8a) wherein all the symbols are as defined earlier.

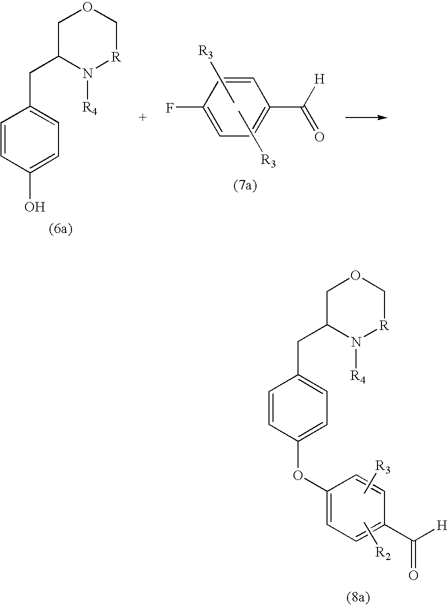

d) Reaction of the compound of formula (8a) with a compound of the formula (9a) gave the compound of formula (10a) wherein all the symbols are as defined earlier.

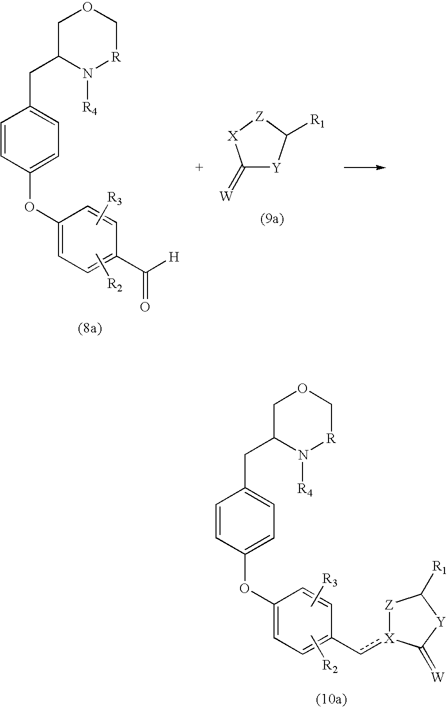

f) Alternatively deprotection or reduction of the compound of formula (10a), wherein R₄ may be the protecting group as defined earlier, produces the compound of formula (I). The order of deprotection and reduction can be changed or reversed.

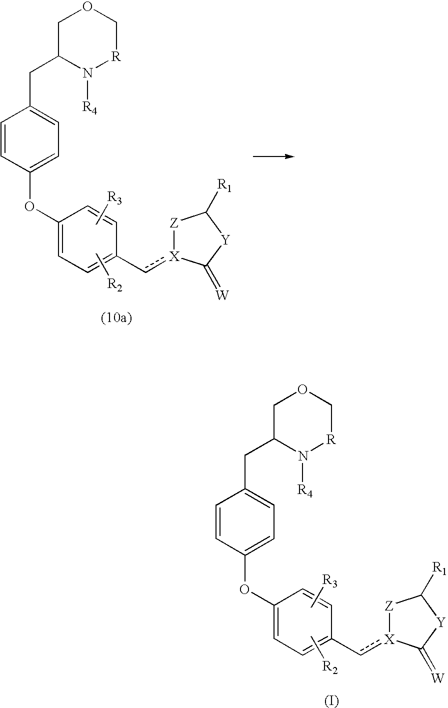

### The Reactions Described in the Processes Outlined Above are Performed by Using the Methods Described Herein:

The deprotection of the compound of formula (1a) to compound of formula (2a) may be carried out using acids such as HCl, sulfuric acid, acetic acid in the presence of solvents such as dichloromethane, ethyl acetate, water and the like or a mixture thereof at a temperature in the range of −10° C. to 50° C.

The reaction of the compound of formula (2a) with chloro acetylchloride is carried out in the presence of solvents such as dichloromethane, tetrahydrofuran, dimethylformamide, dimethyl sulfoxide, DME and the like or a mixtures of solvents may be used to produce the compound of formula (3a). The reaction may be carried out in an inert atmosphere, and may be effected in the presence of a base such as triethylamine, K₂CO₃, Na₂CO₃, NaH or mixtures thereof. The reaction temperature may range from 0° C. to 50° C., preferably in the range of 0° C. to 10° C. The duration of the reaction may range from 1 to 12 hours, preferably from 2 to 6 hours.

Cyclisation of the compound of formula (3a) is carried out in the presence of a base such as potassium t-butoxide, NaH and in the presence of a solvent such as t-butanol, isopropanol, toluene, methoxyethanol or mixtures thereof to yield a compound of formula (4a). The reaction temperature may range from 0° C. to 50° C., preferably in the range of 10° C. to 40° C. The duration of the reaction may range from 1 to 12 hours, preferably from 2 to 6 hours.

Debenzylation of the compound of formula (4a) to the compound of formula (5a) may be carried out in the presence of gaseous hydrogen and a catalyst such as Pd/C, Rh/C, Pt/C, Raney Nickel, and the like. Alternatively mixtures of catalysts may be used. The reaction may be conducted in the presence of solvents such as methanol, dichloromethane, dioxane, acetic acid, ethyl acetate and the like or even mixtures of solvents may be used. A pressure between atmospheric pressure to 100 psi may be employed. The catalyst may be 5-10% Pd/C and the amount of catalyst used may range from 50-300% w/w.

Reduction of the compound (5a) to the compound (6a) may be carried out in the presence of catalyst such as NaBH₄, LiAlH₄ zinc-mercury amalgam, hydrazine and the like. The reaction may be conducted in the presence of solvents such as methanol, dichloromethane, dioxane, acetic acid, ethyl acetate and the like or even a mixture of solvents may be used.

The reaction of the compound of formula (6a) with the compound of formula (7a) is carried out in the presence of solvents such as tetrahydrofuran, dimethylformamide, dimethyl sulfoxide, DME and the like or a mixture of solvents may also be used, to produce the compound of formula (8a). The reaction may be carried out in an inert atmosphere and may be effected in the presence of a base such as K₂CO₃, Na₂CO₃, NaH or mixtures thereof. The reaction temperature may range from 60° C. to 150° C., preferably in the range of 80° C. to 100. ° C. The duration of the reaction may range from 1 to 24 hours, preferably from 2 to 6 hours.

The reaction of the compound of the formula (8a) with a compound of formula (9a) is carried out in the presence of a base using solvents such as toluene, methoxyethanol or mixtures thereof to yield a compound of formula (10a). The reaction temperature may range from 60° C. to 150° C. Suitable catalyst such as piperidinium acetate or benzoate, sodium acetate or a mixture of catalysts may also be employed. The water produced in the reaction may be removed by using Dean Stark water separator or by using water-absorbing agents like molecular sieves.

The deprotection of formula (10a) to yield a compound of formula (I) may be carried out using acids such as HCl, sulfuric acid, acetic acid in the presence of solvents such as dichloromethane, ethyl acetate, water and the like or a mixture thereof at a temperature in the range of −10° C. to 50° C.

In another embodiment of the present invention, there is provided a process for the preparation of compounds of formula (1), by reducing the penultimate step of formula (1) wherein --- represents bond The reduction step is not required when -------- represents no bond and all other symbols are as defined earlier. The reduction may be carried out in the presence of gaseous hydrogen and a catalyst such as Pd/C, Rh/C, Pt/C, Raney Nickel, and the like. A mixture of catalysts may also be used. The reaction may be conducted in the presence of solvents such as methanol, dichloromethane, dioxane, acetic acid, ethyl acetate and the like. Mixtures of solvents may also be used. A pressure between atmospheric pressure to 100 psi may be employed. The catalyst may be 5-10% Pd/C and the amount of catalyst used may range from 50-300% w/w. It may also be noted that the order of deprotection and reduction can be changed or reversed.

It is appreciated that in any of the above-mentioned reactions, any reactive group in the substrate molecule may be protected according to the conventional chemical practice. Suitable protecting groups in any of the above-mentioned reactions are those used conventionally in the art. The methods of formation and removal of such protecting groups are those conventional methods appropriate to the molecule being protected. More specifically the protecting groups P used particularly in the present invention are conventional protecting groups such as t-butoxy carbonyl(t-Boc), trityl, trifluoroacetyl, benzyloxy, benzyloxy carbonyl(Cbz) and the like and deprotection can be done by conventional methods.

The pharmaceutically acceptable salts are prepared by reacting the compound of formula (I) with 1 to 4 equivalents of a base such as sodium hydroxide, sodium methoxide, sodium hydride, potassium t-butoxide, calcium hydroxide, magnesium hydroxide and the like, in solvents like ether, THF, methanol, t-butanol, dioxane, isopropanol, ethanol etc. Mixtures of solvents may also be used. Organic bases like lysine, arginine, diethanolamine, choline, guanidine and their derivatives etc. may also be used. Alternatively, acid addition salts are prepared by treatment with acids such as hydrochloric acid, hydrobromic acid, nitric acid, sulfuric acid, phosphoric acid, p-toluenesulfonic acid, methanesulfonic acid, acetic acid, citric acid, maleic acid, salicylic acid, hydroxynaphthoic acid, ascorbic acid, palmitic acid, succinic acid, benzoic acid, benzene sulfonic acid, tartaric acid and the like in solvents like ethyl acetate, ether, alcohols, acetone, THF, dioxane etc. Mixture of solvents may also be used.

The invention is explained in details in the examples given below which are provided by the way of illustration only and therefore should not be construed to limit the scope of the invention.

### EXAMPLE 1

### Synthesis of 5-[4-(4-{[5-oxomorpholin-3-yl]methyl}phenoxy)benzylidene]-1,3-thiazolidine-2,4-dione

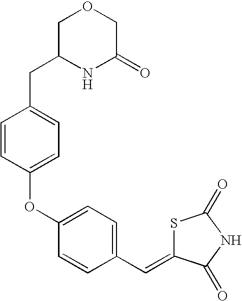

### Step I

### Synthesis of 2-amino-3-[4-(benzyloxy)phenyl]propan-1-ol hydrochloride

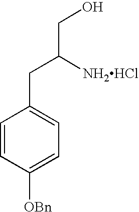

Dry HCl gas was bubbled into a solution of tert-butyl-2-hydroxy-1-(4-benzyloxybenzyl)ethylcarbamate (4g, 11.20 mmol) in dichloromethane (50 ml) at 0-5° C. for two hours. After completion of the reaction, the excess of HCl gas was removed by nitrogen gas bubbling and the white solid thus obtained was filtered and dried to yield 2-amino-3-[4-(benzyloxy)phenyl]propan-1-ol hydrochloride (2.35 g). ¹HNMR [CDCl₃ 400 MHz] δ (ppm): 2.46 (q, 1H), 2.73 (dd, 1H), 3.06 (m, 1H), 3.35 (m, 1H), 3.63 (dd, 1H) 5.04 (s, 2H), 6.91 (d, 2H), 7.11 (d, 2H), 7.40 (m, 5H); MS (ESI, +ve) m/z^{M+1} 259.4

### Step II

### Synthesis of N-{1-[4-(benzyloxy)benzyl]-2-hydroxyethyl}-2-chloroacetamide

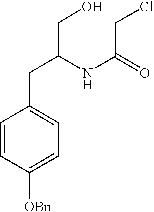

To the suspension of 2-amino-3-[4-(benzyloxy)phenyl]propan-1-ol hydrochloride (1.0 g, 3.40 mmol) in dichloromethane (30 ml) was added triethylamine (1.42 ml, 10.22 mmol) at 0-5° C. followed by chloro acetyl chloride (0.325 ml, 4.04 mmol). After completion of reaction the reaction mixture was washed with 5% aq HCl solution, followed by brine solution, dried over sodium sulfate and concentrated to afford the title compound (0.820 g). ¹HNMR [CDCl₃ 400 MHz]: δ (ppm): 2.85 (m, 2H), 3.63 (dd, 1H), 3.69 (dd, 1H), 4.01 (d, 2E), 4.12 (m, 1H) 5.04 (s, 2H), 6.85 (d, 1H), 6.93 (d, 2H), 7.15 (d, 2H), 7.40 (m, 5H); MS (ESI, +ve) m/z (relative intensity, %): 334.1 (M⁺, 100), 336.1 (M⁺, 33).

### Step III

### Synthesis of 5-[4-(benzyloxy)benzyl]morpholin-3-one

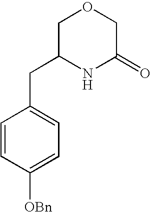

A solution of N-{1-[4-(benzyloxy)benzyl]-2-hydroxyethyl}-2-chloroacetamide (0.8 g, 2.40 mmol) in 30 ml of t-butanol was added to a suspension of potassium t-butoxide (0.4 g, 3.60 mmol) in t-butanol (20 ml) in 10 minutes at 30° C. and stirred for four hours. After completion of reaction, the reaction mixture was quenched with 5% aq. HCl solution and concentrated. The sticky mass thus obtained was neutralized by 5% NaHCO₃ and extracted with ethyl acetate. The organic layer thus obtained was dried over anhydrous sodium sulfate and concentrated to afford the title compound (0.7 g). ¹HNMR [CDCl₃ 400 MHz] δ (ppm): 2.62 (m, 1H), 2.84 (dd, 1H), 3.55 (m, 1H), 3.70 (m, 1H), 3.92 (dd, 1H), 4.16 (d, 2H), 5.06 (s, 2H), 5.84 (bs, 1H), 6.94 (d, 2H), 7.11 (d, 2H), 7.42 (m, 5H); MS (ESI, +ve) m/z^{M+1} 298.3

### Step IV

### Synthesis of 5-(4-hydroxybenzyl)morpholin-3-one

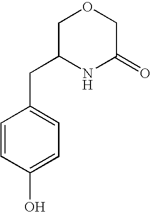

To the solution of 5-[4-(benzyloxy)benzyl]morpholin-3-one (0.7 g, 2.35 mmol) in methanol (100 ml) was added 10% Pd/C (0.100 g). The reaction mixture was hydrogenated at 40 psi for 5-6 hours. The progress of reaction was monitored by TLC. On completion, the solvent was evaporated under reduced pressure to afford the product as an off white solid (0.41 g). ¹HNMR [CDCl₃ 400 MHz] δ (ppm): 2.63 (m, 1H), 2.81 (dd, 1H), 3.54 (m, 1H), 3.68 (m, 1H), 3.89 (dd, 1H), 4.16 (d, 2H), 5.86 (bs, 1H), 6.81 (d, 2H), 7.05 (d 2H); MS (ESI, +ve) m/z^{M+1} 208.3

### Step V

### Synthesis of 4-(4-{[5-oxomorpholin-3-yl]methyl}phenoxy)benzaldehyde

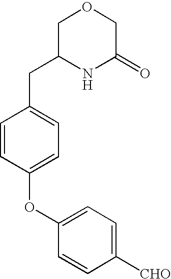

To a suspension of potassium carbonate (1.06 g, 7.68 mmol) in dry DMF (20 ml), was charged 5-(4-hydroxybenzyl)morpholin-3-one (0.4 g, 1.93 mmol), at 30° C. and the reaction mixture was stirred for 15 minutes. Subsequently p-fluorobenzaldehyde (0.239 g, 1.92 mmol) was charged to the reaction mixture, which was warmed to 80° C. and then stirred for 24 hours. The reaction mixture was quenched with water and extracted with ethyl acetate; the combined organic layer was dried over sodium sulfate, concentrated and purified to afford the title compound (0.45 g). ¹HNMR [CDCl₃ 400 MHz] δ (ppm): 2.78 (m, 1H), 2.94 (dd, 1H), 3.61 (m, 1H), 3.68 (m, 1H), 3.92 (dd, 1H), 4.20 (d, 2H), 5.99 (bs, 1H), 7.06 (d, 4H), 7.24 (d, 2H), 7.87 (d, 2H), 9.93 (s, 1H); MS (ESI, +ve) m/z^{M+1} 312.1

### Step VI

### Synthesis of 5-[4-(4-{[5-oxomorpholin-3-yl]methyl}phenoxy)benzylidene]-1,3-thiazolidine-2,4-dione

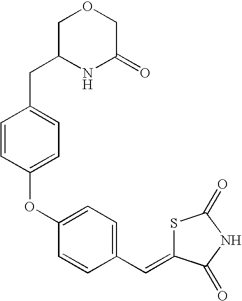

To a suspension of 4-(4-{[5-oxomorpholin-3-yl]methyl}phenoxy)benzaldehyde (0.2 g, 0.643 mmol) in toluene (40 ml) was charged 2,4-thiazolidinonedione (0.090 g, 0.771 mmol), benzoic acid (0.012 g, 0.095 mmol) and piperidine (0.007 g, 0.083 mmol). The reaction mixture was refluxed at 145° C.-155° C. with continuous removal of water using a dean stark apparatus for 3 hours. The solvent was removed by distillation and the crude product thus obtained was purified to yield the product (0.8 g, (30.4%). ¹HNMR [DMSO-d₆ 400MHz] δ (ppm): 2.76 (m, 1H), 2.86 (m, 1H), 3.44 (m, 1H), 3.65 (m, 2H), 3.95 (d, 2H), 7.0 (m, 4H), 7.3 (d, 2H), 7.6 (d, 1H), 7.76 (s, 1H) 8.14 (s, 1H), 12.6 (bs, 1H); MS (ESI, +ve) m/z^{M+1} 411.1

The following compounds were prepared according to the procedure give in
the example 1:

| Example | Structure | Analytical Data |
|---|---|---|
| 2 | 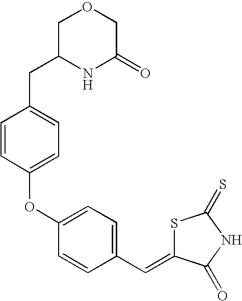 | ¹HNMR [CDCl₃ 400 MHz] δ (ppm): 2.7 (m,1 H), 2.9 (m, 1 H), 3.61 (m, 1 H), 3.75 (m, 1 H),3.94 (dd, 1 H) 4.19 (s, 2 H), 7.05 (m, 4 H), 7.23(d, 2 H), 7.46 (d, 2 H), 7.59 (s, 1 H); MS (ESI,+ve) m/z^{M+1} 427.1 |
| 3 | 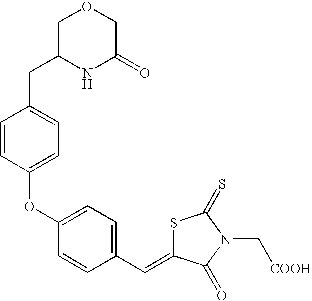 | ¹HNMR [CDCl₃ 400 MHz] δ (ppm): 2.8 (m,2 H), 3.61 (m, 1 H), 3.73 (m, 1 H), 3.88 (m, 1 H),4.17 (s, 2 H), 4.85 (s, 2 H) 7.06 (m, 4 H), 7.24(d, 2 H), 7.49 (d, 2 H), 7.75 (s, 1 H); MS (ESI,+ve) m/z^{M+1} 485.2 |
| 4 | 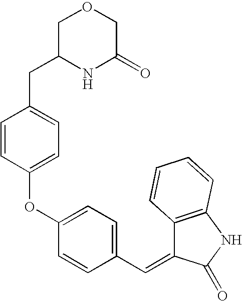 | ¹HNMR [CDCl₃ 400 MHz] δ (ppm): 2.74 (m,1 H), 2.9 (m, 1 H), 3.59 (m, 1 H), 3.77 (m, 1 H),3.94 (dd, 1 H), 4.19 (s, 2 H), 6.11 (s, 1 H), 6.84(d, 1 H), 7.0 (m, 5 H), 7.2 (m, 3 H), 7.52 (d,2 H), 7.84 (s, 1 H), 8.33 (d, 2 H); MS (ESI,+ve) m/z^{M+1} 427.2 |
| 5 | 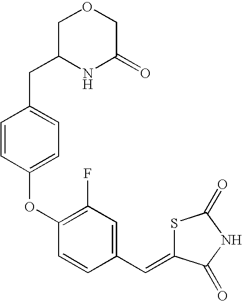 | ¹HNMR [DMSO-d₆, 400 MHz] δ (ppm): 2.76(m, 1 H), 2.85 (m, 1 H), 3.0 (m, 1 H), 3.62 (m,2 H), 3.94 (d, 2 H), 7.03 (d, 2 H), 7.18 (m, 1 H),7.25 (d, 2 H), 7.39 (d, 1 H), 7.64 (dd, 1 H),7.73 (s, 1 H), 8.12 (s, 1 H); MS (ESI, +ve)m/z^{M+1} 429.2 |
| 6 | 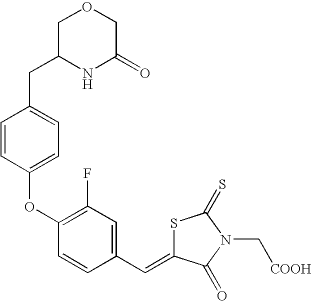 | ¹HNMR [CDCl₃ 400 MHz] δ (ppm): 2.8 (m,2 H), 3.6 (m, 1 H), 3.73 (m, 1 H), 3.8 (dd, 1 H),4.17 (s, 2 H), 4.85 (s, 2 H) 7.0 (m, 3 H), 7.2 (m,3 H), 7.3 (m, 1 H), 7.69 (s, 1 H); MS (ESI, +ve)m/z^{M+} 503.1 |
| 7 | 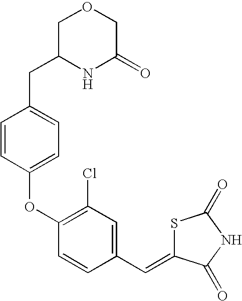 | ¹HNMR [DMSO-d₆, 400 MHz] δ (ppm): 2.75(m, 1 H), 2.88 (m, 1 H), 3.44 (m, 1 H), 3.64 (m,2 H), 3.95 (s, 2 H), 7.08 (m, 3 H), 7.26 (d, 2 H), 7.51(d, 1 H), 7.75 (s, 1 H), 7.86 (s, 1 H), 8.13 (s, 1 H),12.5 (bs, 1 H); MS (ESI, +ve) m/z (relativeintensity, %): 445(M⁺, 100), 447(M⁺, 33). |
| 8 | 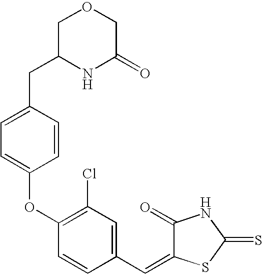 | ¹HNMR [DMSO-d₆, 400 MHz] δ (ppm): 2.75(m, 1 H), 2.88 (m, 1 H), 3.44 (m, 1 H), 3.65 (m,2 H), 3.95 (s, 2 H), 7.06 (m, 3 H), 7.27 (d, 2 H),7.50 (d, 1 H), 7.63 (s, 1 H), 7.88 (s, 1 H), 8.11(s, 1 H), 13.5 (bs, 1 H);MS (ESI, −ve) m/z^{[M-H]}^{^{−}} 458.9. |
| 9 | 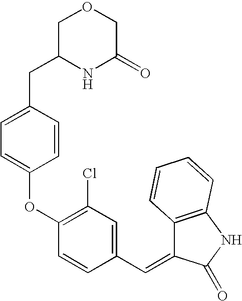 | ¹HNMR [CDCl3, 400 MHz] δ (ppm): 2.76(m, 1 H), 2.9 (m, 1 H), 3.61 (m, 1 H), 3.76 (m,1 H), 3.93 (m, 1 H), 4.19 (s, 2 H), 6.93 (m, 2 H),7.03 (m, 3 H), 7.23 (m, 3 H), 7.42 (d, 1 H),7.51 (s, 1 H), 7.68 (s, 1 H), 7.78 (s, 1 H), 8.2 (d,1 H); MS (ESI, +ve) m/z^{M+1} 461.1 |
| 10 | 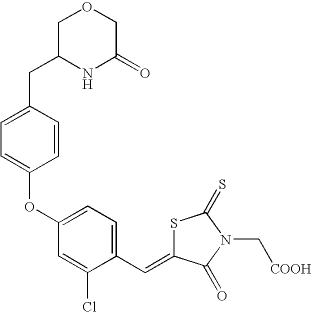 | ¹HNMR [DMSO-d₆, 400 MHz] δ (ppm): 2.8(d, 1 H), 2.91 (d, 1 H), 3.49 (d, 1 H), 3.66 (m,2 H), 3.97 (s, 2 H), 4.72 (s, 2 H), 7.1 (m, 3 H),7.23 (s, 1 H), 7.32 (d, 2 H), 7.61 (d, 1 H), 7.93(s, 1 H), 8.2 (s, 1 H); MS (ESI, +ve) m/z^{M+1}519.0 |
| 11 | 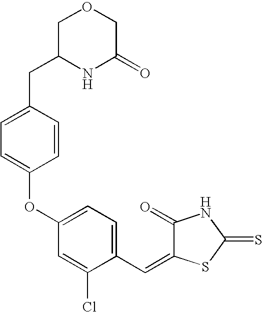 | ¹HNMR [DMSO-d₆, 400 MHz] δ (ppm): 2.78(m, 1 H), 2.89 (m, 1 H), 3.48 (m, 1 H), 3.66 (m,2 H), 3.95 (s, 2 H), 7.1 (m, 3 H), 7.24 (s, 1 H),7.3 (d, 2 H), 7.53 (d, 1 H), 7.69 (s, 1 H), 8.13(s, 1 H), 13.9 (bs, 1 H);MS (ESI, −ve) m/z^{[M-H]}^{^{−}} 458.9. |
| 12 | 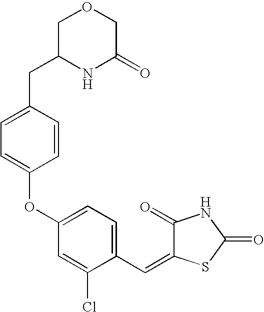 | ¹HNMR [DMSO-d₆, 400 MHz] δ (ppm): 2.77(m, 1 H), 2.88 (m, 1 H), 3.46 (m, 1 H), 3.68 (m,2 H), 3.98 (d, 2 H), 7.1 (m, 3 H), 7.24 (d, 1 H),7.3 (d, 2 H), 7.56 (d, 1 H), 7.88 (s, 1 H), 8.13(s, 1 H), 12.7 (bs, 1 H);MS (ESI, −ve) m/z^{[M-H]}^{^{−}} 442.9 |
| 13 | 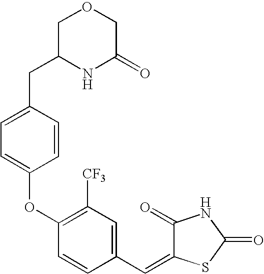 | ¹HNMR [DMSO-d₆, 400 MHz] δ (ppm): 2.78(m, 1 H), 2.88 (m, 1 H), 3.45 (m, 1 H), 3.67 (m,2 H), 3.95 (s, 2 H), 7.1 (m, 3 H), 7.3 (d, 2 H),7.78 (m, 1 H), 7.88 (s, 1 H), 8.06 (s, 1 H), 8.12(s, 1 H), 12.6 (bs, 1 H);MS (ESI, −ve) m/z^{[M-H]}^{^{−}} 477.0 |
| 14 | 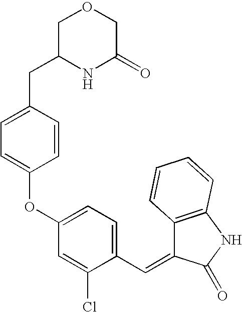 | ¹HNMR [DMSO-d₆, 400 MHz] δ (ppm):2.78(m, 1 H), 2.8 (m, 1 H), 3.46 (m, 1 H), 3.67(m, 2 H), 3.96 (s, 2 H), 6.86 (m, 2 H), 7.12 (m,3 H), 7.23 (m, 1 H), 7.30 (m, 3 H), 7.53 (s,1 H), 7.8 (d, 1 H), 8.1 (s, 1 H), 10.63 (s, 1 H);m/z (relative intensity, %): 461.1 (M⁺, 100),463 (M⁺, 33). |
| 15 | 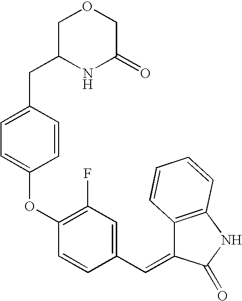 | ¹HNMR [DMSO-d₆, 400 MHz] δ (ppm):2.73(m, 1 H), 2.88 (m, 1 H), 3.45 (m, 1 H),3.65 (m, 2 H), 3.95 (s, 2 H), 6.91 (m, 1 H), 7.04(m, 1 H), 7.20 (m, 2 H), 7.27 (m, 3 H), 7.58 (d,1 H), 7.7 (m, 1 H), 7.8 (d, 1 H), 8.0 (m, 1 H),8.14 (s, 1 H); MS (ESI, +ve) m/z^{M+1} 445.1 |
| 16 | 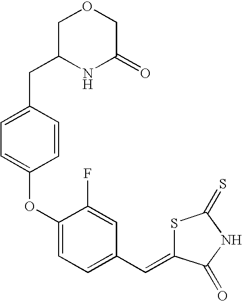 | ¹HNMR [DMSO-d₆, 400 MHz] δ (ppm):2.77(m, 1 H), 2.8 (m, 1 H), 3.43 (m, 1 H), 3.64(m, 2 H), 3.95 (s, 2 H), 7.05 (d, 2 H), 7.17 (d,1 H), 7.26 (d, 2 H), 7.39 (d, 1 H), 7.63 (s, 1 H),7.69 (d, 1 H), 8.12 (s, 1 H), 13.8 (bs, 1 H);MS (ESI, −ve) m/z^{[M-H]}^{^{−}} 443.0 |
| 17 | 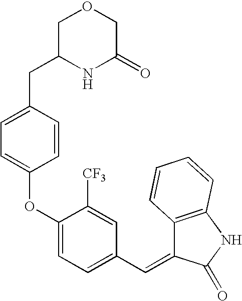 | ¹HNMR [DMSO-d₆, 400 MHz] δ (ppm):2.78(m, 1 H), 2.86 (m, 1 H), 3.4 (m, 1 H), 3.66(m, 2 H), 3.96 (s, 2 H), 6.9 (m, 2 H), 7.0 (m,1 H), 7.11 (m, 3 H), 7.26 (m, 1 H), 7.33 (m,2 H), 7.63 (d, 1 H), 7.85 (m, 1 H), 8.13 (dd,1 H); MS (ESI, −ve) m/z^{[M-H]}^{^{−}} 493.0 |
| 18 | 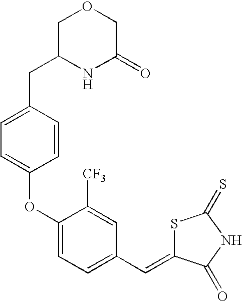 | ¹HNMR [DMSO-d₆, 400 MHz] δ (ppm): 2.78(m, 1 H), 2.86 (m, 1 H), 3.46 (m, 1 H), 3.65 (m,2 H), 3.96 (s, 2 H), 7.10 (m, 3 H), 7.31 (d, 2 H),7.7 (s, 1 H), 7.76 (d, 1 H), 8.1 (dd, 2 H), 13.5(bs, 1 H); MS (ESI, −ve) m/z^{[M-H]}^{^{−}} 493.0 |
| 19 | 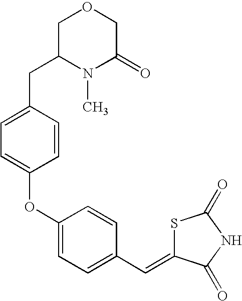 | ¹HNMR [DMSO-d₆, 400 MHz] δ (ppm):2.83 (m, 1 H), 2.91 (s, 3 H), 3.1 (m, 1 H), 3.5(m, 1 H), 3.62 (m, 2 H), 4.03 (s, 2 H), 7.08 (m,4 H), 7.34 (d, 2 H), 7.61 (d, 2 H), 7.74 (s, 1 H);MS (ESI, +ve) m/z^{M+1} 425.0 |
| 20 | 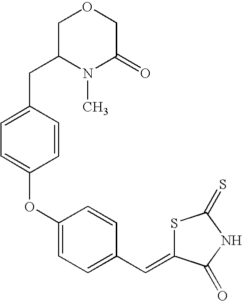 | ¹HNMR [DMSO-d₆, 400 MHz] δ (ppm):2.82 (m, 1 H), 2.91 (s, 3 H), 3.1 (m, 1 H), 3.33(m, 1 H), 3.63 (m, 2 H), 4.04 (s, 2 H), 7.08 (m,4 H), 7.34 (d, 2 H), 7.62 (d, 3 H), 13.8 (s, 1 H);MS (ESI, +ve) m/z^{M+1} 441.0 |
| 21 | 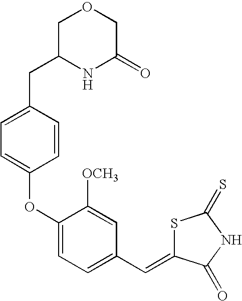 | ¹HNMR [DMSO-d₆, 400 MHz] δ (ppm): 2.72(m, 1 H), 2.85 (m, 1 H), 3.46 (m, 1 H), 3.64 (m,2 H), 3.84 (s, 3 H) 3.97 (s, 2 H), 6.91 (d, 2 H),7.03 (d, 1 H), 7.21 (m, 3 H), 7.34 (s, 1 H), 7.6(s, 1 H), 8.1 (s, 1 H), 13.5 (bs, 1 H);MS (ESI, −ve) m/z^{[M-H]}^{^{−}} 455.0 |
| 22 | 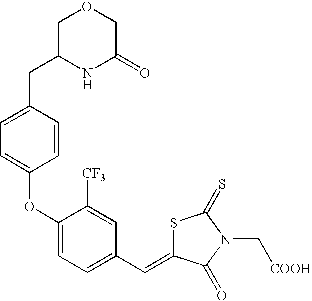 | ¹HNMR [DMSO-d₆, 400 MHz] δ (ppm):2.78 (m, 1 H), 2.90 (m, 1 H), 3.45 (m, 1 H),3.47 (m, 2 H), 3.97 (s, 2 H), 4.65 (s, 2 H), 7.12(m, 3 H), 7.32 (d, 2 H), 7.85 (d, 1 H), 7.98 (s,1 H), 8.15 (dd, 2 H);MS (ESI, −ve) m/z^{[M-H]}^{^{−}} 551.0 |
| 23 | 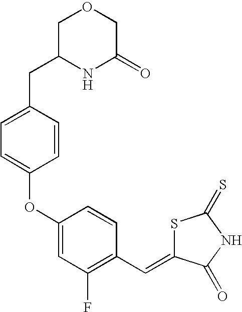 | ¹HNMR [DMSO-d₆, 400 MHz] δ (ppm):2.75 (m, 1 H), 2.84 (m, 1 H), 3.4 (m, 1 H), 3.64(m, 2 H), 3.95 (s, 2 H), 7.03 (d, 2 H), 7.19 (d,1 H), 7.25 (d, 2 H), 7.40 (d, 1 H), 7.64 (d, 1 H),7.71 (s, 1 H), 8.11 (s, 1 H), 12.5 (bs, 1 H);MS (ESI, −ve) m/z^{[M-H]}^{^{−}} 427.0 |
| 24 | 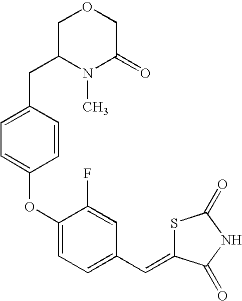 | ¹HNMR [DMSO-d₆, 400 MHz] δ (ppm): 2.8(m, 1 H), 2.91 (s, 3 H), 3.13 (m, 1 H), 3.51 (m,1 H), 3.62 (m, 2 H), 4.04 (m, 2 H), 6.93 (m,1 H), 7.03 (dd, 1 H), 7.13 (d, 2 H), 7.35 (m,2 H), 7.54 (m, 1 H), 7.72 (s, 1 H), 12.4 (bs,1 H); MS (ESI, +ve) m/z^{M+1} 443.0 |
| 25 | 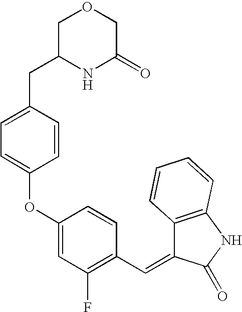 | ¹HNMR [CDCl3, 400 MHz] δ (ppm): 2.77(m, 1 H), 2.89 (m, 1 H), 3.61 (m, 1 H), 3.77 (m,1 H), 3.92 (m, 1 H), 4.19 (s, 2 H) 6.45 (dd, 1 H),6.89 (m, 2 H), 7.03 (m, 4 H), 7.22 (m, 3 H),7.44 (m, 1 H), 7.51 (m, 1 H), 7.68 (m, 1 H),8.48 (m, 1 H); MS (ESI, +ve) m/z^{M+1} 445.1 |
| 26 | 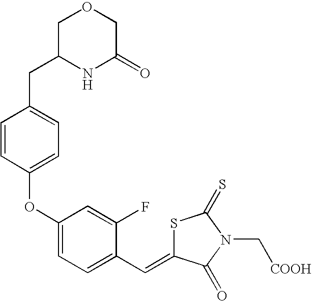 | ¹HNMR [DMSO-d₆, 400 MHz] δ (ppm):2.78 (m, 1 H), 2.88 (m, 1 H), 3.49 (m, 1 H),3.65 (m, 2 H), 3.98 (s, 2 H), 4.67 (s, 2 H), 7.07(d, 2 H), 7.19 (m, 1 H), 7.29 (m, 3 H), 7.46 (d,1 H), 7.7 (d, 1 H), 7.82 (s, 1 H);MS (ESI, −ve) m/z^{[M-H]}^{^{−}} 501.0 |
| 27 | 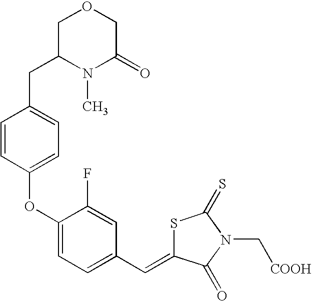 | ¹HNMR [CDCl3, 400 MHz] δ (ppm): 3.0 (m,1 H), 3.06 (s, 3 H), 3.1 (m, 1 H), 3.34 (dd, 1 H),3.68 (dd, 1 H), 3.78 (dd, 1 H), 4.17 (dd, 1 H),4.27 (dd, 1 H), 4.88 (s, 2 H), 6.71 (dd, 1 H),6.86 (dd, 1 H), 7.06 (d, 2 H), 7.27 (d, 1 H), 7.30(d, 1 H), 7.41 (m, 1 H), 7.94 (s, 1 H);MS (ESI, +ve) m/z^{M+1} 517.0 |
| 28 | 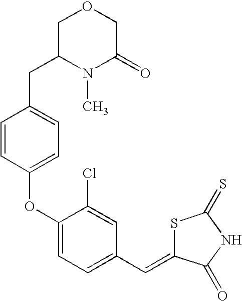 | ¹HNMR [CDCl3, 400 MHz] δ (ppm): 2.97(m, 1 H), 3.1 (m, 4 H), 3.35 (dd, 1 H), 3.67 (dd,1 H), 3.77 (dd, 1 H), 4.25 (q, 2 H), 6.93 (d, 1 H),7.02 (m, 2 H), 7.23 (m, 1 H), 7.30 (m, 2 H), 7.5(m, 2 H); MS (ESI, −ve) m/z^{[M-H]}^{^{−}} 473.3 |
| 29 | 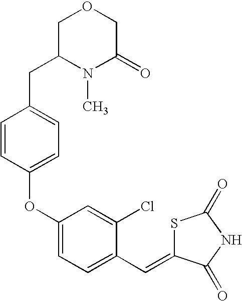 | ¹HNMR [DMSO-d₆, 400 MHz] δ (ppm):2.84 (m, 1 H), 2.91 (s, 3 H), 3.1 (m, 1 H), 3.53(m, 1 H), 3.63 (m, 2 H), 4.03 (s, 2 H), 7.1 (m,3 H), 7.21 (d, 1 H), 7.35 (d, 2 H), 7.58 (d, 1 H),7.84 (s, 1 H), 12.6 (bs, 1 H);MS (ESI, −ve) m/z^{[M-H]}^{^{−}} 457.2 |
| 30 | 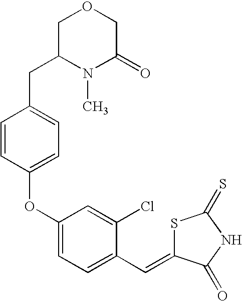 | ¹HNMR [DMSO-d₆, 400 MHz] δ (ppm): 3.0(m, 5 H), 3.13 (m, 1 H), 3.7 (m, 2 H), 4.2 (m,2 H), 6.95 (d, 1 H), 7.05 (d, 2 H), 7.2 (m, 3 H),7.41 (d, 1 H), 7.99 (s, 1 H), 9.5 (bs. 1 H);m/z (relative intensity, %): 474.6 (M⁺, 100),476.7 (M⁺, 33). |
| 31 | 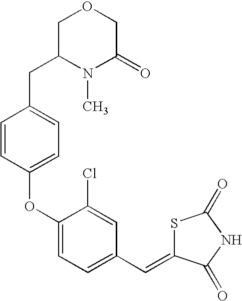 | ¹HNMR [CDCl3, 400 MHz] δ (ppm): 3.0(m, 4 H), 3.33 (m, 2 H), 3.7 (m, 2 H), 4.2 (m,2 H), 7.0 (m, 3 H), 7.23 (m, 2 H), 7.32 (m, 1 H),7.6 (s, 1 H), 7.74 (s, 1 H)MS (ESI, −ve) m/z^{[M-H]}^{^{−}} 456.8 |

### EXAMPLE 32

### 5-[4-(4-{[5-oxomorpholin-3-yl]methyl}phenoxy)benzyl]-1,3-dihydro-2H-indol 2-one

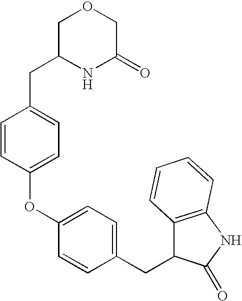

To the solution of 5-[4-(4-{[5-oxomorpholin-3-yl]methyl}phenoxy)benzylidene]-1,3-dihydro-2H-indol-2-one (0.28 g, 0.657 mmol) in methanol (100 ml) was added 10% Pd/C (0.150 g), and the reaction mixture was hydrogenated at 150 psi for 2-3 hours. On completion of the reaction, as monitored by TLC, the solvent was evaporated under reduced pressure to afford the product as an off white solid (0.034 g). ¹HNMR [CDCl₃ 400 MHz] δ (ppm): 2.6 (m, 1H), 2.85 (m, 1H), 3.0 (m, 1H), 3.4 (m, 1H), 3.5 (m, 1H), 3.74 (m, 2H), 3.94 (m, 1H), 4.18 (s, 2H), 5.8 (s, 1H), 6.79 (d, 1H), 6.80 (m, 2H), 6.9 (m, 3H), 7.1 (m, 5H), 7.32 (m, 1H); m/z^{M+1} 429.1

The following compound was prepared according to the procedure give in the example 32:

| Example | Structure | Analytical data |
|---|---|---|
| 33 | 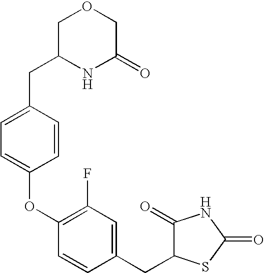 | ¹HNMR [DMSO-d₆, 400 MHz] δ(ppm): 2.71 (m, 1 H), 2.79 (m, 1 H),3.2 (m, 1 H), 3.4 (m, 2 H), 3.62 (m,2 H), 3.94 (m, 2 H), 4.93 (m, 1 H), 6.9(d, 2 H), 7.0 (d, 2 H), 7.2 (d, 2 H),7.29 (d, 1 H), 8.12 (s, 1 H), 12.0 (bs,1 H); MS (ESI, +ve) m/z^{M+1} 431.0 |

### EXAMPLE 34

### 5-(4-{2-chloro-4-[(4-oxo-2-thioxo-1,3-thiazolidin-5-yl)methyl]phenoxy}benzyl) morpholin-3-one

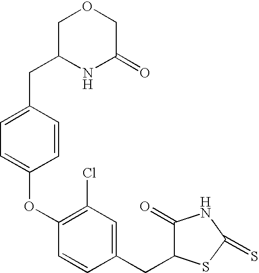

To the solution of 5-(4-{2-chloro-4-[(4-oxo-2-thioxo-1,3-thiazolidin-5-ylidene)methyl]phenoxy}benzyl)morpholin-3-one (0.5 g, 1.08 mmol) in toluene (50 ml) was added 1,4-dihydro-3,5-dicarbethoxy-2,6-dimethyl pyridine (0.35 g, 1.41 mmol) and silica gel 60-120 (1.5 g). The reaction mixture was stirred for 24 hr. at 80-85° C. The progress of reaction was monitored by TLC. After completion of reaction solvent was evaporated under reduced pressure to yield crude product which was purified by column chromatography to gave desired product (0.225 g). ¹HNMR [CDCl3, 400 MHz] δ (ppm): 2.6 (m, 1H), 2.87 (m, 1H), 3.2 (m, 1H), 3.5 (m, 1H), 3.61 (m, 1H), 3.8 (m, 1H), 3.93 (m, 1H), 4.19 (s, 2H), 4.57 (m, 1H), 6.1 (s, 1H), 6.9 (m, 3H), 7.08 (d, 1H), 7.16 (d, 2H), 7.34 (s, 1H), 10.0 (bs, 1H); MS (ESI, −ve) m/z^{[M-H]}^{^{−}} 460.9

### Protocols for Biological Testing

Glucose Uptake Assay Using 3T3-L1 cells

3T3-L1 cells were differentiated by the addition of differentiation cocktail (72 μg/ml insulin, 0.5 mM IBMX, 400 ng/ml Dexamethasone) for 4 days and were later fed with media without differentation cocktail for 7-8 days. After differentiation the cells were incubated with either the reference compound BLX-1002 or the compounds listed in the table 1 at 1 μM concentrations for 72 hours and the glucose uptake assay was carried out for 10 minutes by the addition of KRP buffer supplemented with 2.5 μCi/ml ¹⁴C deoxy glucose. Stimulation index is defined as the amount of ¹⁴C Deoxyglucose uptake induced by 1 μM of BLX-1002 incubated for 72 hours in an assay condition as per the protocol described above with differentiated 3T3-L1 adipocytes. The values for the compounds mentioned in the table-1 are with reference to the stimulation index of the reference compound BLX-1002.

TABLE 1
Effect of compounds on glucose uptake assay in 3T3-L1 cells

| | Stimulation |
|---|---|
| Example No | Index |
| BLX-1002 | 1.00 |
| 1 | 0.93 |
| 2 | 0.89 |
| 3 | 0.92 |
| 4 | 0.99 |
| 5 | 0.91 |
| 6 | 0.94 |
| 7 | 0.85 |
| 8 | 0.87 |
| 10 | 0.89 |
| 11 | 1.05 |
| 12 | 0.96 |
| 13 | 1.14 |
| 14 | 1.11 |
| 17 | 1.09 |
| 19 | 0.99 |
| 20 | 0.96 |
| 34 | 0.89 |

### DPP IV Assay

DPP IV assay is carried by using human plasma as a source of DPP IV. The compounds were incubated at a concentration of 1 and 10 μM in an assay buffer containing the DPP IV enzyme for 1 hr, and then the substrate H-gly-pro AMC was added and further incubated for 20 minutes. Subsequently the reaction was stopped on addition of 25% glacial acetic acid. The plates were read in a spectrofluorimeter to get RFU on setting the excitation wavelength of 360 nm and emission wavelength of 460 nm. The percentage inhibition is calculated as compared to the vehicle control. The results are shown in the table-2, all the compounds studied did not produce a significant DPP IV inhibition.

TABLE 2
DPP IV inhibition of compounds

% DPP IV
Inhibition

| Example No | 1 μM | 10 μM |
|---|---|---|
| 9 | 6.3 | 7.5 |
| 15 | 12.1 | 9.1 |
| 18 | 7.3 | 11.8 |
| 19 | 8.9 | 15.2 |

### Antidiabetic Activity in Streptozotocin Induced Diabetic Mice

Female Swiss albino mice, at the age of 10 weeks were used in the study. Diabetes was induced in the animals by injecting streptozotocin by i.p. route at a dose of 200-mg/kg-body weight. 48 hours after streptozotocin administration, the animals were kept fasting for 6 hours. Subsequently blood was collected, plasma separated and the glucose was estimated. Animals showing greater than 200 mg/dl glucose levels were considered as diabetic and these animals were randomly distributed into various groups. The compounds 2 listed in the table 3 were administered at a dose of 50-mg/kg body weight by oral route for 7 days. Later the animals were fasted for 6 hours, the blood was collected and the plasma was separated. Biochemical estimations like glucose, cholesterol and triglycerides were carried out using the plasma. The effect of the compounds mentioned in the table was expressed in terms of percentage reduction in biochemical values as compared to the control group. The results are as shown in the table-3.

TABLE 3
Effect of compounds in Streptozotocin induced diabetic mice model

% Reduction

| Example No | Glucose | Triglyceride |
|---|---|---|
| 1 | 41.6 | NR |
| 2 | 42.6 | 44.4 |
| 7 | 44.5 | 60.9 |

1. Novel heterocyclic derivatives of the general formula (I) 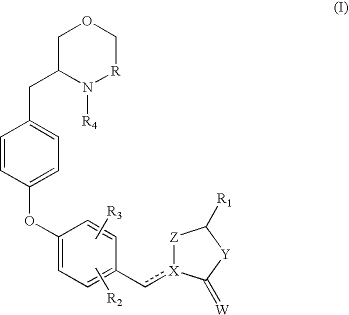
their pharmaceutically acceptable salts and compositions, their analogs, their tautomeric forms, and their stereoisomers; wherein ---- represents an optional bond; R represents CH₂, C═O; W represents O or S; X represents C, CH or N; Y represents NR₅, S or O, wherein R₅ represents hydrogen, substituted or unsubstituted alkyl, alkenyl, —CH₂OOOR′, aryl, or a counter ion; wherein R′ represents H or an alkyl group; Z represents CR₆ or S; R₁ represents ═O, ═S or together with R₆ forms a fused 5 or 6 membered aromatic or heteroaromatic ring system containing carbon atoms or 1 or 2 heteroatoms selected from O, S or N; R₂ and R₃, may be same or different and they independently represent hydrogen, halogen, hydroxy, nitro, cyano, formyl, amino, alkyl, haloalkyl, alkoxy group; R₄ represents H, COR₇, substituted or unsubstituted groups selected from alkyl, alkenyl, aryl, aryloxy, alkoxy, heteroaryl or heterocyclyl; wherein R₇ represents H, substituted or unsubstituted groups selected from alkyl, alkenyl, aryl, aryloxy, alkoxy or aralkoxy.

2. Novel Heterocyclic derivatives as claimed in the claim 1 are selected from a group comprising of:
5-(4-{4-[(5-oxomorpholin-3-yl)methyl]phenoxy}benzylidene)-1,3-thiazolidine-2,4-dione;
5-(4-{4-[(4-oxo-2-thioxo-1,3-thiazolidin-5-ylidene)methyl]phenoxy}benzyl)morpholin-3-one;
5-(-4-{4-[(5-oxomorpholin-3-yl)methyl]phenoxy}benzylidene)-4-oxo-2-thioxo-1,3-thiazolidin-3-yl]acetic acid
5-(4-{4-[(5-oxomorpholin-3-yl)methyl]phenoxy}benzylidene)-1,3-dihydro-2H-indol-2-one
5-(3-fluoro-4-{4-[(5-oxomorpholin-3-yl)methyl]phenoxy}benzylidene)-1,3-thiazolidine-2,4-dione
5-(3-fluoro-4-{4-[(5-oxomorpholin-3-yl)methyl]phenoxy}benzylidene)-4-oxo-2-thioxo-1,3-thiazolidin-3-yl]acetic acid
5-(3-chloro-4-{4-[(5-oxomorpholin-3-yl)methyl]phenoxy}benzylidene)-1,3-thiazolidine-2,4-dione;
5-(4-{2-chloro-4-[(4-oxo-2-thioxo-1,3-thiazolidin-5-ylidene)methyl]phenoxy}benzyl)morpholin-3-one;
5-(3-chloro-4-{4-[(5-oxomorpholin-3-yl)methyl]phenoxy}benzylidene)-1,3-dihydro-2H-indol-2-one
5-(2-chloro-4-{4-[(5-oxomorpholin-3-yl)methyl]phenoxy}benzylidene)-4-oxo-2-thioxo-1,3-thiazolidin-3-yl]acetic acid
5-(4-{3-chloro-4-[(4-oxo-2-thioxo-1,3-thiazolidin-5-ylidene)methyl]phenoxy}benzyl)morpholin-3-one;
5-(2-chloro-4-{4-[(5-oxomorpholin-3-yl)methyl]phenoxy}benzylidene)-1,3-thiazolidine-2,4-dione;
5-[4-{4-[(5-oxomorpholin-3-yl)methyl]phenoxy}-3-(trifluoromethyl)benzylidene]-1,3-thiazolidine-2,4-dione;
5-(2-chloro-4-14-[(5-oxomorpholin-3-yl)methyl]phenoxy}benzylidene)-1,3-dihydro-2H-indol-2-one
5-(3-fluoro-4-{4-[(5-oxomorpholin-3-yl)methyl]phenoxy}benzylidene)-1,3-dihydro-2H-indol-2-one
5-(4-{2-fluoro-4-[(4-oxo-2-thioxo-1,3-thiazolidin-5-ylidene)methyl]phenoxy}benzyl)morpholin-3-one;
5-(3-(trifluoromethyl)-4-{4-[(5-oxomorpholin-3-yl)methyl]phenoxy}benzylidene)-1,3-dihydro-2H-indol-2-one
5-(4-{4-[(4-oxo-2-thioxo-1,3-thiazolidin-5-ylidene)methyl]-2-(trifluoromethyl)phenoxy}benzyl)morpholin-3-one;
5-(4-{4-[(4-methyl-5-oxomorpholin-3-yl)methyl]phenoxy}benzylidene)-1,3-thiazolidine-2,4-dione;
4-methyl-5-(4-{4-[(4-oxo-2-thioxo-1,3-thiazolidin-5-ylidene) methyl]phenoxy}benzyl)morpholin-3-one;
5-(4-{2-methoxy-4-[(4-oxo-2-thioxo-1,3-thiazolidin-5-ylidene)methyl]phenoxy}benzyl)morpholin-3-one;
5-(3-trifluoromethyl-4-{4-[(5-oxomorpholin-3-yl)methyl]phenoxy}benzylidene)-4-oxo-2-thioxo-1,3-thiazolidin-3-yl]acetic acid
5-(4-{3-fluoro-4-[(4-oxo-2-thioxo-1,3-thiazolidin-5-ylidene)methyl]phenoxy}benzyl)morpholin-3-one;
5-(3-fluoro-4-{4-[(4-methyl-5-oxomorpholin-3-yl)methyl]phenoxy)benzylidene)-1,3-thiazolidine-2,4-dione;
5-(2-fluoro-4-{4-[(5-oxomorpholin-3-yl)methyl]phenoxy}benzylidene)-1,3-dihydro-2H-indol-2-one
5-(2-fluoro-4-{-44 (5-oxomorpholin-3-yl)methyl]phenoxy}benzylidene)-4-oxo-2-thioxo-I,3-thiazolidin-3-yl]acetic acid
5-(3-fluoro-4-{4-[(4-methyl-5-oxomorpholin-3-yl)methyl]phenoxy}benzylidene)-4-oxo-2-thioxo-1,3-thiazolidin-3-yl]acetic acid
5-(4-{2-chloro-4-[(4-oxo-2-thioxo-1,3-thiazolidin-5-ylidene)methyl]phenoxy}benzyl)-4-methylmorpholin-3-one;
5-(2-chloro-4-{4-[(4-methyl-5-oxomorpholin-3-yl)methyl]phenoxy}benzylidene)-1,3-thiazolidine-2,4-dione;
5-(4-{3-chloro-4-[(4-oxo-2-thioxo-1,3-thiazolidin-5-ylidene)methyl]phenoxy}benzyl)-4-methylmorpholin-3-one;
5-(3-chloro-4-{4-[(4-methyl-5-oxomorpholin-3-yl)methyl]phenoxy}benzylidene)-1,3-thiazolidine-2,4-dione;
5-(4-{4-[(5-oxomorpholin-3-yl)methyl]phenoxy}benzyl)-1,3-dihydro-2H-indol-2-one
5-(3-fluoro-4-{4-[(5-oxomorpholin-3-yl)methyl]phenoxy}benzyl)-1,3-thiazolidine-2,4-dione;
5-(4-{2-chloro-4-[(4-oxo-2-thioxo-1,3-thiazolidin-5-yl)methyl]phenoxy}benzyl)morpholin-3-one.

3. The compound as claimed in the claim 1, wherein the said pharmaceutically acceptable salt is selected from the group consisting of hydrochloride, hydrobromide, sodium, potassium or magnesium.

4. A pharmaceutical composition, which comprises of a pharmaceutically effective amount of a novel heterocyclic derivative of the formula (I) 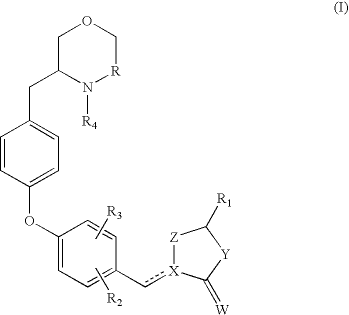
as defined in the claim 1 and a pharmaceutically acceptable carrier, diluent, excipient or solvate.

5. A pharmaceutical composition as claimed in the claim 4, in the form of a tablet, capsule, powder, syrup, solution, aerosol or suspension.

6. A pharmaceutical composition, which comprises of a pharmaceutically effective amount of a novel heterocyclic derivative of the formula (I) 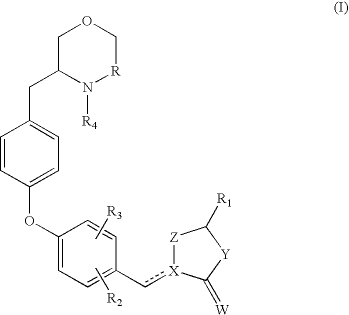
as defined in the claim 1 and a pharmaceutically acceptable carrier, diluent, excipient or solvate, wherein the amount of the compound of claim 1 in the composition is less than 60% by weight.

7. A method for reducing blood glucose, free fatty acids, cholesterol, triglycerides levels in the plasma comprising, administration of an effective amount of a compound of formula (1) as defined in the claim 1 to a patient in need thereof.

8. A method for treating obesity, autoimmune diseases, inflammation, immunological diseases, and cancer disease comprising, administration of an effective amount of a compound of formula (1) as defined in the claim 1 to a patient in need thereof.

9. A method for treating a disorder associated with insulin resistance comprising administration of an effective amount of a compound of formula (1) as defined in the claim 1 to a patient in need thereof.

10. A method for reducing blood glucose levels in the plasma without adipogenic potential comprising, administration of an effective amount of a compound as claimed in the claim 1 to a mammal in need thereof.

11. A method for reducing TNF alfa, IL-6 and IL-beta comprising administration of an effective amount of a compound as claimed in the claim 1 to a mammal in need thereof.

12. A method for reducing cancer cell progression comprising administration of an effective amount of a compound as claimed in the claim 1 to a mammal in need thereof.

13. A method for reducing blood glucose levels in the plasma without adipogenic potential comprising, administration of an effective amount of a compound as claimed in the claim 2 to a mammal in need thereof.

14. A method for reducing TNF alfa, IL-6 and IL-beta comprising administration of an effective amount of a compound as claimed in the claim 2 to a mammal in need thereof.

15. A method for reducing cancer cell progression comprising administration of an effective amount of a compound as claimed in the claim 2 to a mammal in need thereof.
